(19) United States
(12) Patent Application Publication
(10) Pub. No.: US 2018/0057423 A1
(43) Pub. Date: Mar. 1, 2018

(54) DIENE PRODUCTION METHOD

(71) Applicant: JXTG NIPPON OIL & ENERGY CORPORATION, Tokyo (JP)

(72) Inventors: Nobuhiro KIMURA, Tokyo (JP); Junji WAKABAYASHI, Tokyo (JP); Sosuke HIGUCHI, Tokyo (JP)

(73) Assignee: JXTG NIPPON OIL & ENERGY CORPORATION, Tokyo (JP)

(21) Appl. No.: 15/557,640

(22) PCT Filed: Feb. 9, 2016
(86) PCT No.: PCT/JP2016/053797

§ 371 Date: Sep. 12, 2017

(30) Foreign Application Priority Data
Mar. 20, 2015 (JP) 2015-058329

(51) Int. Cl. C07C 5/48; C07C 4/04; C07C 5/25; B01J 23/887; B01J 23/00

(52) U.S. Cl. CPC C07C 5/48; C07C 4/04; C07C 5/2556; B01J 23/8876; B01J 2523/00; C07C 2523/887; C07C 2523/44; C07C 2521/04; C07C 2521/12; B01J 23/002

(57) ABSTRACT

A method for producing diene in which diene can be produced in a high yield by using a raw material including a branched olefin and a straight chain olefin is provided. The method for producing diene comprises: a step 1 of obtaining an internal olefin by removing a branched olefin from a raw material including at least the branched olefin and a straight chain olefin; a step 2 of isomerizing the internal olefin to a terminal olefin by using an isomerization catalyst; and a step 3 of producing diene from the terminal olefin obtained in the step 2 by oxidative dehydrogenation using a dehydrogenation catalyst.

### TECHNICAL FIELD

The present invention relates to a method for producing diene.

### BACKGROUND ART

Dienes such as butadiene are extremely useful as basic raw materials for use in petrochemical industry.

A diene can be obtained by oxidative dehydrogenation of a monoolefin using a dehydrogenation catalyst. Examples of the monoolefin include propylene, 1-butene and 2-butene.

In the oxidative dehydrogenation of a monoolefin, a metal oxide is conventionally used as the dehydrogenation catalyst. As the metal oxide (the dehydrogenation catalyst), for example, a ferrite-based catalyst (see Non Patent Literature 1 mentioned below), a tin-based catalyst (see Non Patent Literature 2 mentioned below) and a bismuth molybdate-based catalyst (see Patent Literatures 1 to 3 and Non Patent Literatures 3 and 4 mentioned below) are known.

### CITATION LIST

### Patent Literature

Patent Literature 1: Japanese Unexamined Patent Publication No. S57-140730
Patent Literature 2: Japanese Unexamined Patent Publication No. S60-1139
Patent Literature 3: Japanese Unexamined Patent Publication No. 2003-220335

### Non Patent Literature

Non Patent Literature 1: J. Catal., 1976, volume 41, 420
Non Patent Literature 2: Petroleum Chemistry U.S.S.R., 1967, 7, 177
Non Patent Literature 3: J. Catal., 1976, 41, 134
Non Patent Literature 4: Handbook of Heterogeneous Catalysis, 1997, 5, 2302

### SUMMARY OF INVENTION

### Technical Problem

As a raw material for use in production of a diene, a raw material including a branched olefin and a straight chain olefin is known. When the raw material including a branched olefin and a straight chain olefin is subjected to oxidative dehydrogenation using a conventional dehydrogenation catalyst (a metal oxide), however, it is difficult to produce a diene in a sufficient yield.

The present invention was accomplished in consideration of the above-described problem, and an object is to provide a method for producing diene in which diene can be produced in a high yield by using a raw material including a branched olefin and a straight chain olefin.

### Solution to Problem

The method for producing diene according to one aspect of the present invention comprises: a step 1 of obtaining an internal olefin by removing a branched olefin from a raw material including at least the branched olefin and a straight chain olefin; a step 2 of isomerizing the internal olefin to a terminal olefin by using an isomerization catalyst; and a step 3 of producing diene from the terminal olefin obtained in the step 2 by oxidative dehydrogenation using a dehydrogenation catalyst.

At least a part of the straight chain olefin may be a terminal olefin, and in the step 1, the branched olefin may be removed from the raw material and the terminal olefin may be isomerized to the internal olefin by reactive distillation.

The isomerization catalyst may include at least one selected from the group consisting of silica and alumina.

The dehydrogenation catalyst may have a complex oxide including bismuth, molybdenum and oxygen.

In the step 2, the internal olefin may be isomerized to the terminal olefin to obtain a first fraction including the terminal olefin and a second fraction including an unreacted portion of the internal olefin by reactive distillation.

In the step 2, the internal olefin may be isomerized to the terminal olefin in a reaction vessel to collect the terminal olefin in the form of a mixture with an unreacted portion of the internal olefin without performing the reactive distillation, and in the step 3, the terminal olefin and the unreacted portion of the internal olefin collected from the reaction vessel may be supplied to the dehydrogenation catalyst.

In the step 3, the diene may be produced from the terminal olefin and the unreacted portion of the internal olefin by using the dehydrogenation catalyst and an isomerization catalyst, and the isomerization catalyst used in the step 3 may include at least one selected from the group consisting of silica and alumina.

Assuming that a mass content of the branched olefin in the raw material is C₁ and a mass content of the straight chain olefin in the raw material is C₂, C₂/C₁ may be 0.1 to 5.0.

The straight chain olefin may include butene.

The raw material may be obtained by fluid catalytic cracking of a heavy oil fraction, and the number of carbon atoms of the branched olefin or the straight chain olefin may be 4.

The raw material may be obtained by thermal decomposition of naphtha, and the number of carbon atoms of the branched olefin or the straight chain olefin may be 4.

### Advantageous Effects of Invention

According to the present invention, diene can be produced in a high yield by using a raw material including a branched olefin and a straight chain olefin.

### DESCRIPTION OF EMBODIMENTS

A preferred embodiment of the present invention will now be described. It is noted that the present invention is not limited to the following embodiment at all.

A method for producing diene according to the present embodiment includes at least a step 1, a step 2 and a step 3.

In the step 1, from a raw material including at least a branched olefin and a straight chain olefin, the branched olefin is removed to obtain an internal olefin. In the step 2, the internal olefin is isomerized by using an isomerization catalyst to produce a terminal olefin. In the step 3, diene is produced from the terminal olefin obtained in the step 2 by oxidative dehydrogenation using a dehydrogenation catalyst. An internal olefin refers to a monoolefin having a double bond in a carbon chain, and is a monoolefin except for a terminal olefin. A terminal olefin refers to a monoolefin having a double bond at the end of a carbon chain.

According to the method for producing diene of the present embodiment, even if the raw material includes a branched olefin and a straight chain olefin, a diene corresponding to the straight chain olefin can be obtained in a high yield. In other words, the yield of diene in oxidative dehydrogenation can be improved.

The yield of diene may be defined, for example, by the following expression 1:

r_{Y1}(%)=m_{P}/m₀₁×100 (1)

wherein m_{P} represents a mass of the diene obtained in the step 3; m₀₁ represents a sum of masses of all hydrocarbons included in the raw material; and r_{Y1} represents a diene yield based on the sum of the masses of all the hydrocarbons included in the raw material.

The diene yield may be defined, for example, in accordance with the following expression 2:

r_{Y2}(%)=m_{P}/m₀₂×100 (2)

wherein m₀₂ represents a sum of masses of all straight chain olefins included in the raw material; and r_{Y2} represents the diene yield based on the sum of the masses of all the straight chain olefins included in the raw material.

The oxidative dehydrogenation of a monoolefin proceeds, for example, through the following reaction path: First, the monoolefin comes into contact with and adsorbs onto a metal oxide (a dehydrogenation catalyst). Next, oxygen in the lattice of the metal oxide pulls out two hydrogen atoms from the adsorbed monoolefin, and thus, the monoolefin is dehydrogenated. As a result, a diene corresponding to the monoolefin and water are produced. Specifically, a diene having the same number of carbon atoms as the monoolefin is produced. After the oxidative dehydrogenation, the resultant oxygen vacancies in the lattice of the metal oxide are filled with molecular oxygen supplied together with the monoolefin.

It is presumed that a high diene yield can be attained in the present embodiment for the following reason:

When a raw material including a branched olefin is subjected to the oxidative dehydrogenation, there may arise problems, for example, that an unwanted byproduct is produced in addition to diene, that the amount of consumed oxygen is increased, and that a dehydrogenation catalyst is deactivated. These problems are suppressed by removing the branched olefin in the step 1. Besides, when in particular a dehydrogenation catalyst including bismuth and molybdenum is used, the oxidative dehydrogenation of the internal olefin obtained in the step 1 is difficult to proceed as compared with the oxidative dehydrogenation of a terminal olefin. This is probably because the internal olefin has a double bond inside a carbon chain, and hence is more difficult to be adsorbed onto the dehydrogenation catalyst than the terminal olefin. Accordingly, when the internal olefin is isomerized to the terminal olefin in the step 2, the oxidative dehydrogenation of the terminal olefin performed in the step 3 is accelerated. In other words, after reducing a ratio of the internal olefin in the straight chain olefin by isomerizing the internal olefin, the oxidative dehydrogenation of the straight chain olefin is performed. In other words, after increasing a ratio of the terminal olefin in the straight chain olefin by isomerizing the internal olefin, the oxidative dehydrogenation of the straight chain olefin is performed. If the step 2 is not performed before supplying the straight chain olefin including the internal olefin to the dehydrogenation catalyst, it is difficult to produce the diene in a sufficient yield. Besides, the oxidative dehydrogenation of the internal olefin is accompanied by a large number of side reactions including a complete oxidation reaction. These side reactions can be suppressed by performing the step 2.

It is presumed that the diene yield is improved through the above-described mechanism. The reason for the improvement of the diene yield is, however, not limited to the above-described reason.

Now, the step 1, the step 2 and the step 3 will be described in detail.

<Step 1>

The raw material used in the step 1 includes a branched olefin and a straight chain olefin. The number of carbon atoms of the branched olefin may be, for example, 4 to 10, or 4 to 6. The number of carbon atoms of the straight chain olefin may be, for example, 4 to 10, or 4 to 6. The number of carbon atoms of the branched olefin may be the same as the number of carbon atoms of the straight chain olefin. The number of carbon atoms of the branched olefin may be different from the number of carbon atoms of the straight chain olefin. The number of carbon atoms of the straight chain olefin may be the same as the number of carbon atoms of a diene to be produced. In other words, the straight chain olefin may be a monoolefin obtained by hydrogenating one of double bonds present in the diene presumed as a product of the step 3.

Assuming that a mass content of all branched olefins in the raw material is C₁ and that a mass content of all straight chain olefins in the raw material is C₂, C₂/C₁ may be 0.1 to 5.0, 0.5 to 5.0, 0.1 to 3.0, or 0.5 to 3.0. In other words, C₂/C₁ may be 0.1 or more, or 0.5 or more. Besides, C₂/C₁ may be 5.0 or less, or 3.0 or less. As C₂/C₁ is larger, the diene yield is more easily increased.

The branched olefin may be, for example, at least one selected from the group consisting of isobutene, 2-methyl-1butene, 2-methyl-2butene, 3-methyl-1butene, 2-methyl-1pentene, 3-methyl-1pentene, 2-methyl-2-pentene and 3-methyl-2-pentene.

The straight chain olefin may be a terminal olefin, or an internal olefin. When an internal olefin is not produced by removing the branched olefin in the step 1, at least a part of the straight chain olefins included in the raw material is an internal olefin. When the branched olefin is removed by, for example, a sulfuric acid absorption process in which a terminal olefin is not isomerized, the raw material originally includes an internal olefin. On the other hand, if an internal olefin is produced from the straight chain olefin by removing the branched olefin, the raw material may originally include a terminal olefin, and need not include an internal olefin. When the branched olefin is removed by, for example, reactive distillation in which a terminal olefin is isomerized, the raw material may originally include a terminal olefin, and need not include an internal olefin. When an internal olefin is produced from a terminal olefin by removing the branched olefin, all the straight chain olefins included in the raw material may be terminal olefins. The raw material may include both a terminal olefin and an internal olefin.

The terminal olefin may be, for example, at least one selected from the group consisting of 1-butene, 1-pentene, 1-hexene, 1-octene and 1-decene. The internal olefin may be, for example, at least one selected from the group consisting of trans-2-butene, cis-2-butene, 2-pentene, 2-hexene, 3-hexene, 2-octene, 3-octene, 4-octene, 2-decene, 3-decene, 4-decene and 5-decene. The raw material may include two or more terminal olefins, and two or more internal olefins.

If the straight chain olefin is butene, the diene yield is easily improved. Specifically, if the internal olefin obtained in the step 1 is 2-butene, 1-butene is obtained as the terminal olefin in the step 2. In the subsequent step 3, 1,3-butadiene is easily obtained in a high yield by the oxidative dehydrogenation of the 1-butene.

The raw material may include, as long as the effects of the present invention are not impaired, an impurity such as hydrogen, carbon monoxide, carbon dioxide gas, water, a saturated hydrocarbon compound, a diene. The saturated hydrocarbon compound may be, for example, at least one selected from the group consisting of methane, ethane, propane, n-butane, cyclobutane and isobutane. If the raw material includes a branched saturated hydrocarbon such as isobutane, the branched saturated hydrocarbon can be removed in the step 1.

The raw material may be a hydrocarbon oil obtained by fluid catalytic cracking of a heavy oil fraction. The number of carbon atoms of a branched olefin or a straight chain olefin included in the hydrocarbon oil may be 4. In other words, the raw material may include a C4 fraction obtained by the fluid catalytic cracking of a heavy oil fraction. The term “C4 fraction” refers to a fraction including, as a principal component, a hydrocarbon having a number of carbon atoms of 4. The raw material may consist of the C4 fraction alone. The C4 fraction may include at least one of 1-butene and 2-butene, and isobutene. If the raw material includes a C4 fraction obtained by the fluid catalytic cracking of a heavy oil fraction, the effects of the present invention are easily obtained. A C4 fraction is comparatively inexpensively available.

The raw material may be a hydrocarbon oil obtained by thermal decomposition of naphtha. The number of carbon atoms of a branched olefin or a straight chain olefin included in the hydrocarbon oil may be 4. In other words, the raw material may be a C4 fraction obtained by the thermal decomposition of naphtha. The raw material may consist of merely a C4 fraction obtained by the thermal decomposition of naphtha. A hydrocarbon oil obtained by separating butadiene from a C4 fraction obtained by the thermal decomposition of naphtha may be used as the raw material. If the raw material includes a C4 fraction obtained by the thermal decomposition of naphtha, the effects of the present invention are easily obtained. A C4 fraction is comparatively inexpensively available:

A method for removing the branched olefin from the raw material in the step 1 is not especially limited. The method for removing the branched olefin from the raw material in the step 1 may be, for example, at least one method selected from the group consisting of reactive distillation (isomerization distillation process), gas adsorption separation process, sulfuric acid absorption process, etherification process and dimerization process. The gas adsorption separation process is a method in which the branched olefin is separated from the raw material by causing the branched olefin included in the raw material in a gas phase to be selectively adsorbed by an adsorbent. The sulfuric acid absorption process is a method in which the branched olefin is separated from the raw material by causing the branched olefin included in the raw material to be selectively absorbed by sulfuric acid. The etherification process is a method in which the branched olefin included in the raw material is reacted with alcohol to form an ether, and the ether is separated from the raw material by distillation. The dimerization process is a method in which the branched olefin included in the raw material is dimerized, and the thus obtained dimer is separated from the raw material by distillation.

If the branched olefin is removed by employing at least one method selected from the group consisting of the gas adsorption separation process, the sulfuric acid absorption process, the etherification process and the dimerization process, the isomerization of the terminal olefin need not be caused in the step 1, and the internal olefin need not be produced. If the terminal olefin is not isomerized in the step 1, the internal olefin obtained in the step 1 is derived from an internal olefin originally included in the raw material.

On the other hand, in employing the reactive distillation (the isomerization distillation process), the branched olefin is removed from the raw material, and in addition, the terminal olefin present in the raw material is isomerized to the internal olefin. Now, the details of the reactive distillation performed in the step 1 will be described.

In the reactive distillation performed in the step 1, an isomerization catalyst is used. This isomerization catalyst has activity to isomerize the terminal olefin included in the raw material to the internal olefin. The isomerization catalyst used in the reactive distillation performed in the step 1 is designated as the “first isomerization catalyst”.

In the reactive distillation performed in the step 1, a distillation column (a first reactive distillation column) in which the first isomerization catalyst is placed is used. In the reactive distillation performed in the step 1, the raw material is supplied to the first reactive distillation column to be brought into contact with the first isomerization catalyst. Thus, the terminal olefin present in the raw material is isomerized to produce the internal olefin. At substantially the same time as the isomerization, the internal olefin and other components derived from the raw material such as the branched olefin are distilled. The boiling point of the internal olefin tends to be higher than the boiling point of the branched olefin. Accordingly, a fraction including the internal olefin (a fraction A) is collected from the bottom of the column by the distillation. On the other hand, a fraction including the branched olefin (a fraction B) is collected from the top of the column.

As described above, in the reactive distillation in step 1, the terminal olefin present in the raw material is isomerized to the internal olefin, and the branched olefin present in the raw material is separated and removed from the other components such as the internal olefin by the distillation. In other words, the isomerization reaction and the distillation are substantially simultaneously performed in the reactive distillation.

If the raw material includes a terminal olefin and a branched olefin having close boiling points, the branched olefin can be easily removed by the reactive distillation performed in the step 1. If the raw material includes, for example, 1-butene and isobutene, the boiling point of 1-butene (−6.6° C. at 1 atm) and the boiling point of isobutene (−6.9° C. at 1 atm) are substantially equivalent. Therefore, it is difficult to separate 1-butene and isobutene from each other by distillation. On the other hand, in the reactive distillation performed in the step 1, 1-butene is isomerized to 2-butene. The boiling point of cis-2-butene (for example, 3.7° C. at 1 atm) and the boiling point of trans-2-butene (for example, 0.9° C. at 1 atm) are both higher than the boiling point of isobutene. Therefore, in the reactive distillation, a fraction including 2-butene (a fraction A) is collected from the bottom of the column, and a fraction including isobutene (a fraction B) is collected from the top of the column.

The temperature in the top of the first reactive distillation column may be adjusted in accordance with the boiling point of the branched olefin. The temperature in the bottom of the first reactive distillation column may be adjusted in accordance with the boiling point of the internal olefin produced from the straight chain olefin. The temperature of the first isomerization catalyst (the reaction temperature of the isomerization) may be adjusted in accordance with the type of the terminal olefin to be isomerized. For example, if 1-butene present in the raw material is to be isomerized to produce 2-butene, the temperature of the first isomerization catalyst (the reaction temperature of the isomerization) may be 20 to 150° C., the air pressure within the first reactive distillation column may be 0 to 5.0 MPaG, and the temperature in the first reactive distillation column top may be 20 to 150° C.

In the reactive distillation performed in the step 1, the raw material may be gasified before being supplied to the first reactive distillation column. Alternatively, the raw material in a liquid form may be supplied to the first reactive distillation column.

The first isomerization catalyst is not especially limited as long as it has activity to isomerize the terminal olefin to the internal olefin. The first isomerization catalyst may include, for example, at least one metal selected from the group consisting of palladium (Pd), nickel (Ni), platinum (Pt), copper (Cu) and silver (Ag). The first isomerization catalyst may be fixed as a catalyst layer in the first reactive distillation column. A reaction vessel filled with the first isomerization catalyst may be placed within the first reactive distillation column.

The fraction A obtained by the reactive distillation performed in the step 1 may include a component except for the internal olefin. For example, the fraction A may include the branched olefin that has not been removed but remains after the step 1. If the fraction A includes the branched olefin, the branched olefin may be removed from the fraction A by supplying the fraction A as the raw material again to the first reactive distillation column. The fraction A may include a hydrocarbon derived from the raw material, or a byproduct of the isomerization reaction. The fraction A may include, for example, hydrogen, carbon monoxide, carbon dioxide gas, methane or a diene.

The number of carbon atoms of the internal olefin obtained in the step 1 may be the same as the number of carbon atoms of the diene of interest. The number of carbon atoms of the internal olefin may be 4 to 10, or 4 to 6.

The internal olefin may be a straight chain unsaturated hydrocarbon. The straight chain unsaturated hydrocarbon may be, for example, at least one selected from the group consisting of trans-2-butene, cis-2-butene, 2-pentene, 2-hexene, 3-hexene, 2-octene, 3-octene, 4-octene, 2-decene, 3-decene, 4-decene and 5-decene.

The internal olefin may have a substituent including a hetero atom such as oxygen, nitrogen, halogen or sulfur. Such a substituent may be, for example, at least one selected from the group consisting of a halogen atom (—F, —Cl, —Br or —I), a hydroxyl group (—OH), an alkoxy group (—OR [wherein R represents a hydrocarbon group]), a carboxyl group (—COOH), an ester group (—COOR [wherein R represents a hydrocarbon group]), an aldehyde group (—CHO) and an acyl group (—C(═O)R [wherein R represents a hydrocarbon group]). The raw material including the internal olefin having the substituent may be, for example, an alcohol, an ether, or a biofuel.

Hereinafter, the hydrocarbon including the internal olefin obtained in the step 1 is designated as the “in-process oil A”. The in-process oil A may consist of the internal olefin alone. The in-process oil A may be the fraction A obtained by the reactive distillation in the step 1. If a mixture including the internal olefin and other components is obtained without performing the reactive distillation in the step 1, the mixture may be used as the in-process oil A. A slight amount of the branched olefin may remain in the in-process oil A. The in-process oil A may include the terminal olefin in addition to the internal olefin.

<Step 2>

In the step 2, the internal olefin obtained in the step 1 is isomerized to a terminal olefin. Specifically, in the step 2, the internal olefin is isomerized by bringing the in-process oil A into contact with an isomerization catalyst to produce a terminal olefin. The isomerization catalyst used in the step 2 is different from the first isomerization catalyst. Hereinafter, the isomerization catalyst used in the step 2 is designated as the “second isomerization catalyst”.

The second isomerization catalyst may include one or a plurality selected from the group consisting of silica, alumina, silica-alumina, zeolite, activated clay, diatomite and kaolin. The second isomerization catalyst may include at least one selected from the group consisting of silica and alumina. If the second isomerization catalyst includes at least one selected from the group consisting of silica and alumina, the internal olefin is easily isomerized in the step 2, and the diene yield is easily improved in the step 3. The second isomerization catalyst may consist of silica-alumina alone.

The second isomerization catalyst may have a support and an element supported on the support (hereinafter sometimes referred to as the “supported element”).

The support may be one or a plurality selected from the group consisting of silica, alumina, silica-alumina, zeolite, activated carbon, activated clay, diatomite and kaolin. The support may include at least one selected from the group consisting of silica and alumina. The support may consist of zeolite alone. A crystalline aluminosilicate generally designated as zeolite has a minute space (a nano-space) of a molecular size in one crystal. The zeolite is classified in accordance with its crystal structure, and there are a large number of types of zeolites such as LTA (A type), MFI (ZSM-5 type), MOR, FER and FAU (X type and Y type) zeolites.

The zeolite may be a faujasite zeolite. The faujasite zeolite is a zeolite expressed as an FAU structure among skeletal structure types in accordance with the IUPAC recommendation. When the second isomerization catalyst has the support including the faujasite zeolite, the internal olefin is easily isomerized in the step 2, and the diene yield is easily improved in the step 3. It is presumed that the second isomerization catalyst including the faujasite zeolite has high isomerization activity because a large amount of supported element (active metal) is highly dispersed in the faujasite zeolite.

The faujasite zeolite may be, for example, at least one selected from the group consisting of X type zeolite, Y type zeolite and USY type zeolite. The faujasite zeolite may be at least one selected from the group consisting of H type, NH₄ type, Na type, Li type, K type, Rb type, Cs type, Fr type, Be type, Mg type, Ca type, Sr type, Ba type and Ra type. Any of these types of faujasite zeolites can be used. The faujasite zeolite may be, for example, at least one selected from the group consisting of HY type zeolite, NH₄Y type zeolite, NaY type zeolite, LiY type zeolite, KY type zeolite, RbY type zeolite, CsY type zeolite, FrY type zeolite, BeY type zeolite, MgY type zeolite, CaY type zeolite, SrY type zeolite, BaY type zeolite, RaY type zeolite, HX type zeolite, NH₄X type zeolite, NaX type zeolite, LiX type zeolite, KX type zeolite, RbX type zeolite, CsX type zeolite, FrX type zeolite, BeX type zeolite, MgX type zeolite, CaX type zeolite, SrX type zeolite, BaX type zeolite and RaX type zeolite. Any of these types of faujasite zeolites can be used. Such a faujasite zeolite can be prepared by, for example, ion exchange of a metal element (a cation) included in the faujasite zeolite. In the present embodiment, when the support includes X type zeolite, the internal olefin is easily isomerized in the step 2 and the diene yield is easily improved in the step 3. Since X type zeolite has a comparatively large number of ion exchange sites, the amount of supported element (for example, the amount of Ag) per unit volume in the X type zeolite can be large. Accordingly, if the X type zeolite is used, the internal olefin is easily isomerized in the step 2, and the diene yield is easily improved in the step 3. A part or the whole of cations (such as H⁺, NH₄⁺, Na⁺, Li⁺, K⁺, Rb⁺, cS⁺, Fr⁺, Be²⁺, Mg²⁺, Ca²⁺, Sr²⁺, Ba²⁺ and Ra²⁺) of the faujasite zeolites may be substituted by the supported element.

The supported element of the second isomerization catalyst may be at least one element selected from the group consisting of Group 10 elements of the periodic table, Group 11 elements of the periodic table, and lanthanoids. The periodic table refers to a long period periodic table of elements defined by IUPAC (International Union Pure and Applied Chemistry). The supported element may be an element except for Group 10 elements of the periodic table and Group 11 elements of the periodic table, and lanthanoids.

Group 10 elements of the periodic table may be, for example, at least one selected from the group consisting of nickel (Ni), palladium (Pd) and platinum (Pt). Group 11 elements of the periodic table may be, for example, at least one selected from the group consisting of copper (Cu), silver (Ag) and gold (Au). The lanthanoids may be, for example, at least one selected from the group consisting of lanthanum (La) and cerium (Ce). The element supported on the support may be a combination of these elements. It is preferable for the element supported on the support to be Ag. When Ag is supported on the support, the internal olefin is easily isomerized in the step 2, and the diene yield is easily improved in the step 3.

In the step 2, the internal olefin may be isomerized to a terminal olefin by reactive distillation using the second isomerization catalyst to obtain a first fraction including the terminal olefin and a second fraction including an unreacted portion of the internal olefin. The reactive distillation performed in the step 2 is different from the reactive distillation performed in the step 1.

In the reactive distillation performed in the step 2, a distillation column (a second reactive distillation column) in which the second isomerization catalyst is placed is used. In the reactive distillation performed in the step 2, the in-process oil A is supplied to the second reactive distillation column to be brought into contact with the second isomerization catalyst. Thus, the internal olefin included in the in-process oil A is isomerized to produce a terminal olefin. At substantially the same time as the isomerization, the terminal olefin and other components derived from the in-process oil A such as an unreacted portion of the branched olefin are distilled. The boiling point of the internal olefin tends to be higher than the boiling point of the terminal olefin. Accordingly, the fraction including the internal olefin (the second fraction) is collected by the distillation from the bottom of the column. On the other hand, the fraction including the terminal olefin (the first fraction) is collected from the top of the column. The second isomerization catalyst may be fixed as a catalyst layer in the second reactive distillation column. A reaction vessel filled with the second isomerization catalyst may be placed within the second reactive distillation column.

For example, if the in-process oil A includes 2-butene as the internal olefin, the 2-butene is isomerized to produce 1-butene as the terminal olefin in the reactive distillation of the step 2. The boiling point of cis-2-butene is 3.7° C. and the boiling point of trans-2-butene is 0.9° C. The boiling points of both the 2-butenes are higher than the boiling point of 1-butene (−6.6° C.). Accordingly, through the reactive distillation of the step 2, the fraction including 2-butene (the second fraction) is collected from the bottom of the column, and the fraction including 1-butene (the first fraction) is collected from the top of the column.

The isomerization of the internal olefin is an equilibrium reaction. In other words, the amount of the terminal olefin relative to that of the internal olefin in an equilibrium state has an upper limit. Accordingly, in the reactive distillation performed in the step 2, the terminal olefin may be continuously distilled off from the top of the column. In this case, the amount of the terminal olefin relative to that of the internal olefin in the second reactive distillation column easily becomes smaller than the amount of the terminal olefin relative to that of the internal olefin in an equilibrium state. Therefore, the internal olefin is easily isomerized in the second reactive distillation column, and hence the terminal olefin is easily produced. As a result, the diene yield is easily improved in the step 3.

The temperature in the top of the second reactive distillation column may be adjusted in accordance with the boiling point of the terminal olefin. The temperature in the bottom of the second reactive distillation column may be adjusted in accordance with the boiling point of the unreacted portion of the internal olefin. The temperature of the second isomerization catalyst (the reaction temperature of the isomerization) may be adjusted in accordance with the type of the internal olefin to be isomerized. For example, if 2-butene included in the raw material is to be isomerized to produce 1-butene, the temperature of the second isomerization catalyst (the reaction temperature of the isomerization) may be 20 to 150° C.

In the reactive distillation performed in the step 2, the in-process oil A may be gasified before being supplied to the second reactive distillation column. Alternatively, the in-process oil A in a liquid form may be supplied to the second reactive distillation column. The second fraction collected from the second reactive distillation column may be supplied again to the second reactive distillation column, so that the internal olefin included in the second fraction may be isomerized to the terminal olefin. As a result, the diene yield is easily improved in the step 3.

The first fraction obtained by the reactive distillation of the step 2 may include a component except for the terminal olefin. For example, a slight amount of an unreacted portion of the internal olefin may remain in the first fraction. The first fraction may include a hydrocarbon derived from the in-process oil A or a byproduct of the isomerization reaction. The first fraction may include, for example, hydrogen, carbon monoxide, carbon dioxide gas, methane or a diene.

In the step 2, the internal olefin may be isomerized to the terminal olefin in a reaction vessel without performing the reactive distillation. In this case, the terminal olefin may be collected in the form of a mixture with an unreacted portion of the internal olefin. For example, the second isomerization catalyst is placed in a reaction vessel (a reaction vessel different from a distillation column). Subsequently, the in-process oil A is supplied to the reaction vessel to be brought into contact with the second isomerization catalyst. Thus, the internal olefin included in the in-process oil. A is isomerized to produce the terminal olefin. Subsequently, a mixture of the terminal olefin and the unreacted portion of the internal olefin is collected from the reaction vessel. The in-process oil A may be supplied into the reaction vessel after being gasified. The in-process oil A in a liquid form may be supplied into the reaction vessel.

As described above, in the step 2, the terminal olefin produced in the reaction vessel and other components derived from the in-process oil A such as the unreacted portion of the internal olefin may be collected from the reaction vessel in the form of a mixture without fractionating.

A reaction system for the isomerization of the internal olefin not by the distillation is not especially limited. The reaction system may be, for example, a fixed bed system, a moving bed system or a fluidized bed system. The reaction vessel may be a flow reaction vessel or a batch reaction vessel.

Hereinafter, the hydrocarbon including the terminal olefin obtained in the step 2 is designated as the “in-process oil B”. The in-process oil B may consist of the terminal olefin alone. The in-process oil B may be the first fraction obtained by the reactive distillation of the step 2. If the mixture including the terminal olefin and the unreacted portion of the internal olefin is obtained in the step 2, the mixture may be used as the in-process oil B.

<Step 3>

In the step 3, diene is produced, by oxidative dehydrogenation using a dehydrogenation catalyst, from the terminal olefin obtained in the step 2. In other words, in the step 3, the in-process oil B including the terminal olefin obtained in the step 2 is brought into contact with a dehydrogenation catalyst to oxidatively dehydrogenate the terminal olefin, and thus, diene is produced.

The dehydrogenation catalyst may have a complex oxide including bismuth (Bi), molybdenum (Mo) and oxygen. If the dehydrogenation catalyst has a complex oxide including bismuth, molybdenum and oxygen, the oxidative dehydrogenation of the terminal olefin is easily accelerated, and the diene yield is easily improved.

The composition of the complex oxide is not especially limited. The complex oxide may consist of merely bismuth, molybdenum and oxygen. The complex oxide may include an additional component in addition to bismuth, molybdenum and oxygen. The additional component may be, for example, at least one selected from the group consisting of cobalt (Co), nickel (Ni), iron (Fe), magnesium (Mg), calcium (Ca), zinc (Zn), cerium (Ce), samarium (Sm), sodium (Na), potassium (K), rubidium (Rb), cesium (Cs), thallium (Tl), boron (B), phosphorus (P), arsenic (As) and tungsten (W).

The dehydrogenation catalyst may consist of merely the complex oxide including bismuth, molybdenum and oxygen. The dehydrogenation catalyst may include a component except for the complex oxide. Besides, the dehydrogenation catalyst may contain a molding aid as long as the physical properties and the catalyst performance of the catalyst are not impaired. The molding aid may be, for example, at least one selected from the group consisting of a thickener, a surfactant, a water retention agent, a plasticizer and a binder material.

In the step 3, a reaction vessel filled with the dehydrogenation catalyst may be used to produce diene by the oxidative dehydrogenation of the terminal olefin.

A reaction system for the oxidative dehydrogenation of the terminal olefin is not especially limited. The reaction system may be, for example, a fixed bed system, a moving bed system or a fluidized bed system. If the oxidative dehydrogenation of the terminal olefin is performed by employing the fixed bed system, the process design can be easily performed.

The oxidative dehydrogenation of the terminal olefin may be a gas phase reaction. Specifically, the in-process oil B including the terminal olefin is first gasified by using a vaporizer or the like. Next, the gaseous in-process oil B and a molecular oxygen-containing gas are heated to about 150 to 250° C. using a preheater or the like, and the resultant gases are supplied into the reaction vessel. In other words, the oxidative dehydrogenation of the terminal olefin may be performed in the presence of the molecular oxygen-containing gas. The in-process oil B and the molecular oxygen-containing gas may be supplied to the reaction vessel after preheating in a mixed state, namely, in the form of a mixed gas. The in-process oil B and the molecular oxygen-containing gas may be separately preheated before being supplied to the reaction vessel through separate tubes. If the in-process oil B and the molecular oxygen-containing gas are mixed to be preheated and then supplied to the reaction vessel, these gases are homogeneously mixed. Therefore, a phenomenon in which heterogeneously mixed gases produce a detonating gas in a reaction vessel is suppressed. Besides, a situation where raw materials having different compositions are supplied through different tubes of a multi-tubular reaction vessel is difficult to occur.

The gaseous in-process oil B and the molecular oxygen-containing gas are supplied to the reaction vessel, and at the same time, a nitrogen gas and water (water vapor) may be supplied to the reaction vessel. By adjusting the amount of the nitrogen gas and water (water vapor) to be supplied, the concentrations of a combustible gas such as the in-process oil B and the molecular oxygen in a gas (a reaction gas) supplied to the reaction vessel can be adjusted. Besides, when water (water vapor) is supplied to the reaction vessel, the coking of the dehydrogenation catalyst is easily suppressed. The nitrogen gas and water (water vapor) may be mixed with the gaseous in-process oil B and the molecular oxygen-containing gas before preheating the gaseous in-process oil B and the molecular oxygen-containing gas. The nitrogen gas and water (water vapor) may be separately preheated before being directly supplied to the reaction vessel through separate tubes.

The composition of the reaction gas may be controlled so that the composition of the reaction gas does no fall in an explosive range at the inlet of the reaction vessel. The composition of the reaction gas may be controlled while monitoring flow rates of the respective gases included in the reaction gas. The flow rates of the respective gases can be monitored, for example, by providing a flowmeter in each tube used for supplying each of the gases. The explosive range refers to a composition range in which a mixed gas (the reaction gas) of oxygen (the molecular oxygen) and a combustible gas (the gaseous in-process oil B) ignites in the presence of some ignition source. Besides, the highest concentration of the combustible gas at which the mixed gas ignites is designated as an upper explosive limit. The lowest concentration of the combustible gas at which the mixed gas ignites is designated as a lower explosive limit. If the concentration of the combustible gas in the mixed gas is higher than the upper explosive limit or lower than the lower explosive limit, the mixed gas does not ignite. Furthermore, an oxygen concentration at which the upper explosive limit and the lower explosive limit have the same value is designated as a limiting oxygen concentration. If the oxygen concentration is lower than the limiting oxygen concentration, the mixed gas does not ignite regardless of the concentration of the combustible gas.

The composition of the reaction gas at the inlet of the reaction vessel and the reaction conditions may be adjusted so that the composition of a product (a product gas) at the outlet of the reaction vessel does not fall in the explosive range. Besides, the composition of the reaction gas at the inlet of the reaction vessel and the reaction conditions may be adjusted so that an oxygen concentration in the product gas can be lower than the limiting oxygen concentration. Specifically, the oxygen flow rate may be adjusted so that the oxygen concentration in the reaction gas can be 11% by volume or less. The oxygen concentration in the reaction gas may be measured with an oxygen analyzer provided at the inlet of the reaction vessel.

At the beginning of the supply of the reaction gas, the composition of the reaction gas may be adjusted so that the oxygen concentration in the reaction gas can be lower than the limiting oxygen concentration. Besides, as the reaction proceeds, the amounts of the material gas and the molecular oxygen-containing gas supplied may be increased so as to adjust the composition of the reaction gas in such a manner that the concentration of the material gas in the reaction gas can be higher than the upper explosive limit.

The temperature within the reaction vessel (the reaction temperature of the oxidative dehydrogenation) is not especially limited. The reaction temperature may be, for example, 280 to 400° C. If the reaction temperature is 280° C. or more, a sufficient diene yield tends to be obtained because equilibrium conversion of the terminal olefin does not become too low. If the reaction temperature is 400° C. or less, high activities of the dehydrogenation catalyst can be easily retained for a long period of time because the coking rate thereof is suppressed.

The pressure within the reaction vessel (the air pressure in the reaction vessel) is not especially limited. The air pressure in the reaction vessel may be, for example, 0 MPaG or more, 0.02 MPaG or more, or 0.05 MPaG or more. As the air pressure in the reaction vessel is higher, the amount of the reaction gas that can be supplied to the reaction vessel is larger. Besides, the air pressure in the reaction vessel may be, for example, 0.5 MPaG or less, 0.3 MPaG or less, or 0.1 MPaG or less. As the air pressure in the reaction vessel is lower, the explosive range tends to be smaller.

The weight hourly space velocity (WHSV) in the oxidative dehydrogenation of the terminal olefin may be 0.01 to 50 h⁻¹, or 0.05 to 10 h⁻¹. Here, the WHSV refers to a ratio (F/W) of a supply rate F (supplied amount/time) of the gaseous in-process oil B to the mass W (catalyst mass) of the dehydrogenation catalyst in a continuous reactor. If the WHSV is 50 h⁻¹ or lower, the terminal olefin included in the gaseous in-process oil B can be brought into contact with the dehydrogenation catalysts for a sufficient time period, and hence, the oxidative dehydrogenation of the terminal olefin can easily proceed. If the WHSV is 0.01 h⁻¹ or higher, the decomposition of a hydrocarbon compound does not excessively proceed, and hence, the efficiency of producing diene is easily improved. Incidentally, the use amounts of the terminal olefin and the dehydrogenation catalyst may be appropriately selected to fall in more preferable ranges in accordance with the reaction conditions, the activity of the catalyst and the like, and the WHSV is not limited to the above-described range.

The content of the molecular oxygen in the molecular oxygen-containing gas may be 10% by volume or more, 15% by volume or more, or 20% by volume or more. Incidentally, from the viewpoint of cost necessary for industrially preparing the molecular oxygen-containing gas, the content of the molecular oxygen in the molecular oxygen-containing gas may be 50% by volume or less, 30% by volume or less, or 1% by volume or less.

The molecular oxygen-containing gas may include an arbitrary impurity as long as the effects of the present invention are not impaired. Such an impurity may be, for example, nitrogen, argon, neon, helium, carbon monoxide, carbon dioxide or water. The molecular oxygen-containing gas may be, for example, air. The content of nitrogen in the molecular oxygen-containing gas may be 90% by volume or less, 85% by volume or less, or 80% by volume or less. The content of an impurity except for nitrogen may be 10% by volume or less, or 1% by volume or less. If the contents of these impurities are too large, there is a tendency that the molecular oxygen in an amount necessary for the reaction is difficult to supply.

As long as the effects of the present invention are not impaired, the dehydrogenation of the terminal olefin can be performed in the presence of the terminal olefin (the in-process oil B), the molecular oxygen-containing gas, nitrogen gas, water (water vapor) and an additional component. The additional component may be, for example, methane, hydrogen or carbon dioxide.

If the in-process oil B used in the step 3 is a mixture including the terminal olefin and an unreacted portion of the internal olefin, diene may be produced from the terminal olefin and the unreacted portion of the internal olefin by using a dehydrogenation catalyst and an isomerization catalyst. The isomerization catalyst to be used together with the dehydrogenation catalyst in the step 3 is designated as the “third isomerization catalyst”. The internal olefin included in the in-process oil B comes into contact with the third isomerization catalyst to be isomerized to a terminal olefin. Subsequently, the terminal olefin comes into contact with the dehydrogenation catalyst to produce diene. In other words, when the dehydrogenation catalyst and the third isomerization catalyst are used together, the diene can be produced not only from the terminal olefin but also from the internal olefin. The third isomerization catalyst may be the same as the second isomerization catalyst.

The dehydrogenation catalyst and the third isomerization catalyst may be separately placed in the reaction vessel. In other words, the reaction vessel may be provided with a catalyst layer including the dehydrogenation catalyst and another catalyst layer including the third isomerization catalyst. Alternatively, a mixture including the dehydrogenation catalyst and the third isomerization catalyst may be used. In other words, the reaction vessel may be provided with a catalyst layer including the dehydrogenation catalyst and the third isomerization catalyst.

A product (a product gas) of the oxidative dehydrogenation may include a component except for the diene of interest. The product of the oxidative dehydrogenation may include, for example, a hydrocarbon derived from the in-process oil B, the dehydrogenation catalyst or a byproduct of the oxidative dehydrogenation. The byproduct of the oxidative dehydrogenation may be, for example, water, an oxygen-containing compound, a light olefin, or an olefin polymer. The byproduct may be, for example, water, an oxygen-containing compound, a light olefin, or an olefin polymer. The oxygen-containing compound may be, for example, carbon monoxide or carbon dioxide. The light olefin may be, for example, ethylene or propylene. Such an impurity may be separated from the product by any known method.

The diene obtained in the step 3 may be, for example, at least one selected from the group consisting of 1,3-butadiene, piperylene, isoprene, 1,5-hexadiene, 1,6-octadiene and 1,9-decadiene. Specifically, if the internal olefin obtained in the step 1 is trans-2-butene or cis-2-butene, 1,3-butadiene is likely to be obtained. If the internal olefin obtained in the step 1 is 2-pentene, piperylene is likely to be obtained. If the internal olefin obtained in the step 1 is 2-hexene or 3-hexene, 1,5-hexadiene is likely to be obtained. According to the method for producing diene of the present embodiment, a thermodynamically stable conjugated diene can be easily obtained.

1,3-Butadiene, that is, a representative example of diene, is used as a raw material of a synthetic rubber such as SBR (styrene-butadiene rubber) or NBR (acrylonitrile-butadiene rubber), or a raw material of an ABS (acrylonitrile butadiene styrene) resin or the like.

According to the present embodiment described so far, even if a raw material including a branched olefin and a straight chain olefin is used, the diene yield is improved as compared with that obtained by a conventional production method.

### EXAMPLES

Now, the present invention will be described in more detail with reference to examples and a comparative example, and it is noted that the present invention is not limited to these examples at all.

(Preparation of Dehydrogenation Catalyst)

A dehydrogenation catalyst to be used in the step 3 was prepared as follows:

To 250 ml of pure water, 54 g of ammonium paramolybdate was added to be dissolved therein by heating to 70° C., and thus, a solution A was obtained. Next, to 60 ml of pure water, 7.18 g of ferric nitrate, 31.8 g of cobalt nitrate and 31.8 g of nickel nitrate were added to be dissolved therein by heating to 70° C., and thus, a solution B was obtained. The solution B was gradually added to the solution A under sufficiently stirring the solution A, and thus, a mixed solution of the solution A and the solution B was obtained. Next, 64 g of silica was added to the thus obtained mixed solution, and the resultant was sufficiently stirred to obtain a slurry A. The slurry A was held at 75° C. for 5 hours. Thereafter, the slurry A was dried by heating, and the resultant was heated at 300° C. for 1 hour under air atmosphere, and thus, a first granular solid (a catalyst precursor) was obtained. The loss-on-ignition of the first granular solid was 1.4% by mass.

A solution C was obtained by mixing 40.1 g of ammonium paramolybdate, 150 ml of pure water and 10 ml of ammonia water. The first granular solid was ground and dispersed in the solution C to obtain a slurry B. Next, 0.85 g of borax and 0.36 g of potassium nitrate were added to 40 ml of pure water to be dissolved therein under heating at 25° C., and thus, a solution D was obtained. The slurry B was added to the solution D, and 58.1 g of bismuth subcarbonate in which 0.45% by mass of Na had been dissolved to form a solid solution was further added thereto, followed by stirring for mixing, and thus, a slurry C was obtained. The slurry C was dried by heating at 130° C. for 12 hours to obtain a second granular solid. The second granular solid was tablet-molded using a small molding machine to obtain a tablet. The tablet had a diameter of 5 mm and a height of 4 mm. The tablet was calcined at 500° C. for 4 hours to obtain a dehydrogenation catalyst made of a complex oxide. An atomic ratio in the dehydrogenation catalyst calculated based on the amounts of fed raw materials is as follows:

### <Atomic Ratio>

Mo:Bi:Co:Ni:Fe:Na:B:K:Si=12:5:2.5:2.5:0.4:0.35:0.2:0.08:24

### Example 1

<Preparation of Raw Material>

A raw material of Example 1 including the following components was prepared. Assuming that a mass content of a branched olefin (isobutene) in the raw material is C₁ and a mass content of straight chain olefins (1-butene, cis-2-butene and trans-2-butene) in the raw material is C₂, C₂/C₁ was 2.6.

Isobutane: 41.0% by mass

Isobutene: 13.0% by mass

1-Butene: 12.0% by mass

Normal butane: 12.0% by mass

Cis-2-butene: 9.0% by mass

Trans-2-butene: 13.0% by mass

<Step 1>

Reactive distillation of the step 1 was performed as follows.

A first isomerization catalyst was fixed in a first reactive distillation column. As the first isomerization catalyst, a catalyst in which 0.3 to 0.4% by mass of Pd was supported on a support of γ alumina was used. The above-described raw material was supplied to the first reactive distillation column to be brought into contact with the first isomerization catalyst. A rate of the raw material fed into the first reactive distillation column was set to 30 t/h. A fraction A was collected from the bottom of the first reactive distillation column, and a fraction B was collected from the top of the first reactive distillation column. A flow rate of the fraction A flowing out from the bottom was 14.1 t/h (corresponding to 47% by mass of the total mass of the raw material).

The thus obtained fraction A was analyzed using a gas chromatograph equipped with a flame ionization detector. Concentrations (in % by mass) of respective components in the fraction A were quantitatively determined by an absolute calibration curve method based on the gas chromatography. The composition of the fraction A (the concentrations of the respective components in the fraction A) is shown in Table 1 below. It is noted that a concentration may be also designated as a mass content (a content).

By the same method as that employed for the fraction A, the composition of the fraction B was analyzed. As a result of the analysis, it was confirmed that the fraction B mostly included isobutane and isobutene.

<Step 2>

The reactive distillation of the step 2 was reproduced by performing, using a computer, a simulation based on a reactive distillation column simulation model. The details of the simulation were as follows:

As simulation software, VMG ver 8.0 manufactured by Vertual Materials Group Inc. was used. The following sub-steps a, b, c and d were reproduced in this order by the simulation. These sub-steps constitute the step 2.

Sub-step a: The second isomerization catalyst was filled in a reaction vessel including ten serially connected complete mixing type tanks.

Sub-step b: The reaction vessel including the complete mixing type tanks of the sub-step a was disposed between the 100th plate and the 20th plate of a second reactive distillation column having 120 theoretical plates.

Sub-step c: The fraction A obtained in the step 1 was supplied to the second reactive distillation column of the sub-step b to produce 1-butene by isomerizing 2-butene included in the fraction A.

Sub-step d: A first fraction was collected from the top of the second reactive distillation column, and a second fraction was collected from the bottom of the second reactive distillation column.

In the simulation, silica-alumina was reproduced as the second isomerization catalyst. As the activity of the silica-alumina, activity of silica-alumina (trade name: “IS-28”) manufactured by JGC Catalysts and Chemicals Ltd. was presumed. Parameters relating to the second isomerization catalyst were input so as to reproduce the presumed activity. In the sub-step c, the composition of the fraction A of Example 1 shown in Table 1 below was input as a reactant of the isomerization reaction. As a physical property estimation equation for the simulation, Advanced Peng-Robinson equation was used.

Values of various parameters employed in the simulation were as follows:

Activity energy of second isomerization catalyst (silica-alumina): 40 kJ/mol

Frequency factor of second isomerization catalyst: 10

Flow rate of fraction A: 30 t/h

Flow rate of first fraction: 20 t/h (corresponding to 35% by mass of total mass of fraction A)

Flow rate (bottom flow) of second fraction: 10 t/h

Bottom temperature: 80.8° C.

Bottom pressure: 1000 KPa

Reflux ratio: 7.5

Number of feed plates: 113

The concentrations (in % by mass) of the respective components in the first fraction of Example 1 calculated through the simulation are shown in Table 1 below.

<Step 3>

A tubular reaction vessel (a tube of SUS) was filled with 17 cc of the dehydrogenation catalyst. The tubular reaction vessel had an inner diameter of 14 mm and a length of 60 cm. The reaction vessel filled with the dehydrogenation catalyst was connected to a flow reactor, and then the temperature within the reaction vessel was increased up to 330° C. by using an electric furnace. The first faction of Example 1 having the composition calculated by the simulation of the step 2 was actually prepared. The first fraction, air and water vapor were supplied to the reaction vessel after the temperature increase, so as to be brought into contact with the dehydrogenation catalyst. In this manner, the oxidative dehydrogenation of the first fraction was performed in the reaction vessel. The rates of the first fraction, the air and the water vapor flowing into the reaction vessel were adjusted respectively to the following values. A Ni content in the dehydrogenation catalyst filled in the reaction vessel was 0.54 g.

Flow rate of first fraction A: 2.16 g/h

Flow rate of air: 60 cc/min

Flow rate of water vapor: 1.5 g/h

When 120 minutes had elapsed from a reaction start time, a product gas was collected from the reaction vessel. It is noted that the time when the first fraction was started to be supplied was regarded as the reaction start time (minute 0). The thus collected product gas was analyzed using a gas chromatograph equipped with a flame ionization detector. Concentrations (in % by mass) of respective components in the product gas were quantitatively determined by the absolute calibration curve method based on the gas chromatography. The concentrations of the respective components in the product gas are shown in Table 1 below. Next, on the basis of the concentrations of the respective components thus determined, butadiene yield R_{Y1}(%) and butadiene yield R_{Y2}(%) were calculated. The yields R_{Y1} and R_{Y2} are shown in Table 1 below. Incidentally, the butadiene yield R_{Y1} is defined in accordance with the following expression 1a. The butadiene yield R_{Y2} is defined in accordance with the following expression 2a.

R_{Y1}=Sw×M_{P}/100 (1a)

M_{P} in the expression 1a represents the concentration (in % by mass) of butadiene included in the product gas. Sw (in parts by mass) represents a relative mass (in parts by mass) of all hydrocarbons included in the product gas assuming that the total mass of all hydrocarbons included in the raw material is 100 parts by mass.

R_{Y2}=[(Sw×M_{P})/(100×M_{b})]×100 (2a)

In the expression 2a, 100 of 100×M_{b} corresponds to the total mass (100 parts by mass) of all the hydrocarbons included in the raw material. M_{b} represents a sum of concentrations (in % by mass) of 1-butene, cis-2-butene and trans-2-butene included in the raw material.

### Example 2

The step 1 of Example 2 was performed in the same manner as in Example 1 except that a raw material of Example 2 having a composition shown in Table 1 below was used, and thus, a fraction A and a fraction B of Example 2 were obtained. The step 2 of Example 2 was performed in the same manner as in Example 1 except that the fraction A of Example 2 was used, and thus, a first fraction of Example 2 was obtained. The step 3 of Example 2 was performed in the same manner as in Example 1 except that the first fraction of Example 2 was used, and thus, a product gas of Example 2 was obtained. In the same manner as in Example 1, the fraction A, the first fraction and the product gas (the product of the step 3) of Example 2 were respectively analyzed. The analysis results of Example 2 are shown in Table 1 below. Yields R_{Y1} and R_{Y2} of Example 2 calculated in the same manner as in Example 1 are shown in Table 1 below. It is noted that the most part of the fraction B of Example 2 was found to be isobutane and isobutene.

### Example 3

The step 1 of Example 3 was performed in the same manner as in Example 1 to obtain a fraction A and a fraction B of Example 3. The fraction A of Example 3 was the same as the fraction A of Example 1. The fraction B of Example 3 was the same as the fraction B of Example 1.

In Example 3, the following step 2 was actually performed without performing the simulation. In the following step 2, however, distillation was not performed.

A tubular reaction vessel (a tube of SUS) was filled with 1.7 cc of silica-alumina (a second isomerization catalyst). As the silica-alumina, IS-28 manufactured by JGC Catalysts and Chemicals Ltd. was used. The tubular reaction vessel had an inner diameter of 14 mm and a length of 60 cm. The reaction vessel filled with the silica-alumina was connected to a flow reactor, and then the temperature within the reaction vessel was increased up to 330° C. by using an electric furnace. The fraction A obtained in the step 1 was supplied to the reaction vessel after the temperature increase. The flow rate of the fraction A was 2.2 g/h. In this manner, cis-2-butene and trans-2-butene included in the fraction A were isomerized to obtain an in-process oil B including 1-butene.

The thus obtained in-process oil B was analyzed using a gas chromatograph equipped with a flame ionization detector. The concentrations (in % by mass) of respective components in the in-process oil B were quantitatively determined by the absolute calibration curve method based on the gas chromatography. The concentrations of the respective components in the in-process oil B are shown in Table 1 below.

The step 3 of Example 3 was performed in the same manner as in Example 1 except that the in-process oil B of Example 3 was used instead of the first fraction. A product gas of Example 3 obtained in the step 3 was analyzed in the same manner as in Example 1. The concentrations of respective components in the product gas of Example 3 are shown in Table 1 below. Yields R_{Y1} and R_{Y2} of Example 3 calculated in the same manner as in Example 1 are shown in Table 1 below.

### Comparative Example 1

The step 1 of Comparative Example 1 was performed in the same manner as in Example 1 to obtain a fraction A and a fraction B of Comparative Example 1. The fraction A of Comparative Example 1 was the same as the fraction A of Example 1. The fraction B of Comparative Example 1 was the same as the fraction B of Example 1. Subsequently, the step 3 of Comparative Example 1 was performed in the same manner as in Example 1 except that the fraction A of Comparative Example 1 was used instead of the first fraction. In other words, the step 2 was not performed but the step 3 was performed subsequently to the step 1 in Comparative Example 1. A product gas obtained in the step 3 of Comparative Example 1 was analyzed in the same manner as in Example 1. The concentrations of respective components of the product gas of Comparative Example 1 are shown in Table 1 below. Yields R_{Y1} and R_{Y2} of Comparative Example 1 calculated in the same manner as in Example 1 are shown in Table 1 below.

TABLE 1

| | | Example 1 | | | | Example 2 | | | |
|---|---|---|---|---|---|---|---|---|---|
| | | | Fraction | First | Product | | Fraction | First | Product |
| | | Material | A | Fraction | Gas | Material | A | Fraction | Gas |
| Composition | Isobutane | 41.0 | 0.0 | 0.0 | 0.0 | 3.4 | 0.0 | 0.0 | 0.0 |
| (% by mass) | Isobutene | 13.0 | 0.5 | 0.8 | 0.0 | 44.0 | 0.8 | 1.2 | 0.0 |
| | 1-Butene | 12.0 | 0.0 | 68.6 | 6.4 | 22.9 | 0.0 | 67.8 | 6.4 |
| | Normal | 12.0 | 27.5 | 27.5 | 27.6 | 14.7 | 27.7 | 27.7 | 27.6 |
| | Butane | | | | | | | | |
| | Cis-2- | 9.0 | 28.0 | 0.5 | 1.8 | 5.0 | 27.8 | 0.5 | 1.8 |
| | butene | | | | | | | | |
| | Trans-2- | 13.0 | 44.0 | 2.7 | 2.4 | 9.9 | 43.7 | 2.8 | 2.4 |
| | butene | | | | | | | | |
| | Butadiene | 0.0 | 0.0 | 0.0 | 61.8 | 0.0 | 0.0 | 0.0 | 61.8 |
| C₂/C₁ | | 2.6 | — | — | — | 0.9 | — | — | — |
| Relative Mass | | 100 | 47 | 31 | 31 | 100 | 53 | 35 | 35 |
| (parts by mass) | | | | | | | | | |
| R_{Y1} (%) | | — | — | — | 19 | — | — | — | 22 |
| R_{Y2} (%) | | — | — | — | 57 | — | — | — | 58 |
| | | | Example 3 | | | | Comparative Example 1 | | |
| | | | | Fraction | in-process | Product | | Fraction | Product |
| | | | Material | A | oil B | Gas | Material | A | Gas |

| | | | | | | | | |
|---|---|---|---|---|---|---|---|---|
| Composition | Isobutane | 41.0 | 0.0 | 0.0 | 0.0 | 41.0 | 0 | 0.0 |
| (% by mass) | Isobutene | 13.0 | 0.5 | 0.0 | 0.0 | 13.0 | 0.5 | 0.0 |
| | 1-Butene | 12.0 | 0.0 | 15.5 | 4.1 | 12.0 | 0 | 2.8 |
| | Normal Butane | 12.0 | 27.5 | 27.6 | 27.6 | 12.0 | 27.5 | 27.6 |
| | Cis-2-butene | 9.0 | 28.0 | 23.5 | 16.6 | 9.0 | 28 | 20.2 |
| | Trans-2-butene | 13.0 | 44.0 | 33.4 | 27.5 | 13.0 | 44 | 33.9 |
| | Butadiene | 0.0 | 0.0 | 0.0 | 24.2 | 0.0 | 0 | 15.5 |

| | | | | | | | |
|---|---|---|---|---|---|---|---|
| C₂/C₁ | 2.6 | — | — | — | 2.6 | — | — |
| Relative Mass | 100 | 47 | 47 | 47 | 100 | 47 | 47 |
| (parts by mass) | | | | | | | |
| R_{Y1} (%) | — | — | — | 11 | — | — | 7 |
| R_{Y2} (%) | — | — | — | 34 | — | — | 22 |

### INDUSTRIAL APPLICABILITY

According to the present embodiment, diene can be mass-produced in a high yield from a raw material including a branched olefin and a straight chain olefin.

1. A method for producing diene, comprising:
a step 1 of obtaining an internal olefin by removing a branched olefin from a raw material including at least the branched olefin and a straight chain olefin;
a step 2 of isomerizing the internal olefin to a terminal olefin by using an isomerization catalyst; and
a step 3 of producing diene from the terminal olefin obtained in the step 2 by oxidative dehydrogenation using a dehydrogenation catalyst.

2. The method for producing diene according to claim 1,
wherein at least a part of the straight chain olefin is a terminal olefin, and
wherein in the step 1, the branched olefin is removed from the raw material and the terminal olefin is isomerized to the internal olefin by reactive distillation.

3. The method for producing diene according to claim 1,
wherein the isomerization catalyst includes at least one selected from the group consisting of silica and alumina.

4. The method for producing diene according to claim 1,
wherein the dehydrogenation catalyst has a complex oxide including bismuth, molybdenum and oxygen.

5. The method for producing diene according to claim 1,
wherein in the step 2, the internal olefin is isomerized to the terminal olefin to obtain a first fraction including the terminal olefin and a second fraction including an unreacted portion of the internal olefin by reactive distillation.

6. The method for producing diene according to claim 1,
wherein in the step 2, the internal olefin is isomerized to the terminal olefin in a reaction vessel to collect the terminal olefin in the form of a mixture with an unreacted portion of the internal olefin without performing reactive distillation, and
wherein in the step 3, the terminal olefin and the unreacted portion of the internal olefin collected from the reaction vessel are supplied to the dehydrogenation catalyst.

7. The method for producing diene according to claim 6,
wherein in the step 3, the diene is produced from the terminal olefin and the unreacted portion of the internal olefin by using the dehydrogenation catalyst and an isomerization catalyst, and
wherein the isomerization catalyst used in the step 3 includes at least one selected from the group consisting of silica and alumina.

8. The method for producing diene according to claim 1,
wherein assuming that a mass content of the branched olefin in the raw material is C₁ and a mass content of the straight chain olefin in the raw material is C₂, C₂/C₁ is 0.1 to 5.0.

9. The method for producing diene according to claim 1,
wherein the straight chain olefin includes butene.

10. The method for producing diene according to claim 1,
wherein the raw material is obtained by fluid catalytic cracking of a heavy oil fraction, and
a number of carbon atoms of the branched olefin or the straight chain olefin is 4.

11. The method for producing diene according to claim 1,
wherein the raw material is obtained by thermal decomposition of naphtha, and
a number of carbon atoms of the branched olefin or the straight chain olefin is 4.
